(19) United States
(12) Patent Application Publication
(10) Pub. No.: US 2004/0204492 A1
(43) Pub. Date: Oct. 14, 2004

(54) Topical composition and method for treating seborrheic dermatitis

(75) Inventors: Braham Shroot, San Antonio, TX (US); Robert Espinoza, San Benito, TX (US); Jimmy Joe Willis, Mansfield, TX (US)

Correspondence Address: MCKEE, VOORHEES & SEASE, P.L.C., 801 GRAND AVENUE, SUITE 3200, DES MOINES, IA, 50309-2721, US

(73) Assignee: Healthpoint, Ltd., Fort Worth, TX

(21) Appl. No.: 10/406,412

(22) Filed: Apr. 3, 2003

(51) Int. Cl. A61K 31/18

(52) U.S. Cl. 514/602

(57) ABSTRACT

A topical composition and method for treating seborrehic dermatitis designed specifically to allow for short duration skin contact time. It is a water rinseable sulfacetamide composition free of elemental sulfur, preferably dosed in a regimen of at least two daily treatments.

### BACKGROUND OF THE INVENTION

Seborrheic dermatitis is a chronic cutaneous inflammatory condition, marked by frequent exacerbations, and affecting areas of skin in which sebaceous glands have a dense distribution and are highly active. Lesions of seborrehic dermatitis are red and scaly and may be marked by intense itching. Facial seborrehic dermatitis is typically associated with greasy scaling of the scalp, with erythema and scaling of the nasolabial folds, ears and other areas of the face including eyebrows, forehead, jaw/cheek area and central chin. Structures associated with the eyes may be involved (blepharitis and occlusion of the meibomian glands). Facial seborrehic dermatitis involvement may be associated with a burning sensation in the affected areas.

Seborrheic dermatitis is one of the most common and most familiar dermatologic disorders. It affects at least 3% of the general population. A sample of elderly patients (mean age 70) disclosed a high prevalence of inflammatory eczema associated with seborrehic dermatitis, exacerbated by frequent bathing and the use of strong soaps. Seborrheic dermatitis, once considered to be a condition primarily of the aged (“senile keratosis”), has been found to have a prevalence of 15.7% in teenagers (15 to 19 years) and 32.3% in young adults (25 to 30 years), independent of sex, skin type, hair or eye color. It is commonly seen in infants, adults between the ages of 30 and 60 and immunocompromised patients.

Because sex hormones play an important role in the production of sebum, the onset of seborrehic dermatitis is usually after puberty, and the condition is more prevalent in men than women, because of androgen stimulation of the human sebocyte. Evidence suggests that seborrehic dermatitis is not simply a result of excessive production of sebum, however. The high prevalence and chronicity of seborrehic dermatitis, along with the still limited understanding of the etiology and pathophysiology of the disease and absence of disease-specific rational therapy have made the management of seborrehic dermatitis a source of frustration for physician and patient alike. Patients with the disease often have very sensitive skin. Treatment of seborrehic dermatitis of the face has depended on the use of antifungal shampoos and topical antifungal or anti-inflammatory agents.

Sodium sulfacetamide, sometimes referred to as sulfacetamide sodium, is a treatment that has been used for seborrehic dermatitis in the past. It has, however, been typically used in topicals that also contain free sulfur as a keratolytic agent, see for example Plexion™ Cleanser which contains sodium sulfacetamide 10% and sulfur 5%. Products containing sulfur are undesirable because of the risk of an adverse reaction. Although the mechanism of sulfur's keratolytic action has not been determined, this action may depend on formation of hydrogen sulfide when the drug comes in contact with skin, which can cause irritation. Other examples are those compositions like the one sold under the trademark Carmol™ which is a scalp treatment lotion containing sodium sulfacetamide 10% designed as a stay on product for prolonged skin contact. Products designed for prolonged disease treatment run the risk of patient adverse reaction to sodium sulfacetamide which will prolong disease treatment, i.e. skin contact can risk an irritation or sensitization, Stevens-Johnson Syndrome in hypersensitive individuals, etc.

It can therefore be seen that there is a need for a safe and effective sodium sulfacetamide product designed specifically for the treatment of seborrheic dermatitis and designed for skin contact for the requisite time for therapeutic effectiveness but not prolonged skin contact such that risk of adverse skin reaction becomes increased. Therefore, a delivery system using sodium sulfacetamide, preferably 10% (w/w), as the active which could be effective in a product designed for easy application and easy rinse off would represent a significant advance in the management of this common and troublesome disorder.

It is a primary objective of this invention to provide such a delivery system which can be effectively used for patients diagnosed with seborrheic dermatitis.

### SUMMARY OF THE INVENTION

A topical composition and method for treating seborrehic dermatitis designed specifically to allow for short duration skin contact time. It is a water rinseable sulfacetamide composition free of elemental sulfur preferably dosed in a regimen of at least two daily treatments. The invention surprisingly provides effectiveness with minimal skin contact to reduce irritation risk.

While the preferred delivery system is a wash, since this is the easiest for quick control and short skin contact time, it may be also formulated into other delivery systems that are water rinseable such as aerosol foams, gels, lotions, soap bars, and creams. The system is designed for skin contact of from ten (10) seconds to two (2) minutes.

### DESCRIPTION OF THE INVENTION

According to this invention, a delivery system for alkaline earth sulfacetamides in general, and in particular sodium sulfacetamide is designed to provide sulfacetamide therapy, controlled so that it has sufficient contact with skin to provide the needed therapeutic activity but to avoid prolonged skin contact and the risk of adverse skin reaction. Some have termed this type of regime as “short contact therapy”. The regime here is specifically designed for a composition that is free of sulfur to further avoid adverse patient skin reaction. It is also designed for ease of water rinse (thereby avoiding prolonged contact period). The typical regime involves two times daily dosing. The typical contact time is from ten (10) seconds to about two (2) minutes, at least once daily, preferably twice daily (morning and evening) or as directed by a treating physician.

Sulfacetamide sodium is C₈H₉N₂NaO₃S.H₂O with a molecular weight of 254.24. Chemically, it is Acetamide N-[(4-aminophenyl)sulfonyl]-, monosodium salt, monohydrate, with the following structural formula:

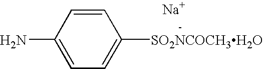

Sulfacetamide sodium is an odorless, white, crystalline powder with a bitter taste. It is freely soluble in water, sparingly soluble in alcohol, while practically insoluble in benzene, in chloroform, and in ether.

Sulfacetamide sodium exerts a bacteriostatic effect against sulfonamide sensitive gram-positive and gram-negative microorganisms commonly isolated from secondary cutaneous phylogenic infections. It acts by restricting the synthesis of folic acid required by bacteria for growth, by its competition with para-aminobenzoic acid.

While the above description has been given with a sodium salt of sulfacetamide, any soluble alkaline earth salt of sulfacetamide may be used. Typically sodium salt is preferred because of ease of availability. The amount employed will vary but generally is within the range of from 2% (w/w) basis to 15% (w/w) of the total composition. The preferred range is from 8% (w/w) basis to 12% (w/w) basis of the composition. The most commonly used dosage, and that known for safety and efficacy, is a 10% (w/w) basis.

It is understood that the above ranges have been given as exemplary only and that the critical factor is use of a “therapeutically effective amount”. As used herein, “therapeutically effective amount” means the amount administered to a human patient to effectively treat seborrehic dermatitis avoiding prolonged skin contact to the point that normal patients have risk of sensitive skin reaction. Normally for the preferred wash composition, the administration regime is as follows. Wash affected areas twice daily (morning and evening), or as directed by your physician. Wet the skin and liberally apply to areas to be cleansed, massage gently into skin for ten (10) seconds to two (2) minutes working into a full lather, rinse thoroughly and pat dry. Rinsing with plain water removes any excess medication. Repeat the application as described for eight to ten days. If needed repeat above application for another 8-10 days. If skin dryness occurs it may be controlled by rinsing cleanser off sooner or using less frequently.

The sulfacetamide composition contains the active sulfacetamide compound of the invention in amounts suitable for topical use on humans in therapeutic effective amounts as previously defined. Such compositions may be in the form of washes, aerosol foams, gels, lotions, soap bars, or creams and can include a variety of preservatives, carriers and other inactive or active ingredients, with the entire composition being water rinseable. The preferred delivery form is a wash which also has cleansing properties. For cleansing washes, the wash may contain a primary cleansing surfactant at a level of from 2% (w/w) to 50% (w/w), preferably 5% (w/w) to 20% (w/w) and most preferred at a level of 12%. The preferred cleaning surfactant, although any which may be usable in a water based composition will do as long as it is not skin sensitive, is sodium laureth sulfate. A secondary surfactant such as a foam booster may also be used and when so used, is present at a level of from 1% (w/w) to 40% (w/w), preferably 3% (w/w) to 15% (w/w) and most preferred 8% (w/w). The preferred secondary surfactant is cocamidopropyl betaine.

Other minors well known to topical formulators may also be employed. Such can include surfactant thickeners at a level from 0.5% (w/w) to 15% (w/w), preferably 1% (w/w) to 8% (w/w) and most preferably at a 5% (w/w) level. One such suitable surfactant thickener is available commercially from Croda, Inc. and is sold as “Crothix Liquid”. Other minors can include emollients/moisturizers for skin-feel softness, antioxidants, preservatives, etc. These are referred to herein as “pharmaceutical minors”, not so much for the amounts they represent which are typically 5% (w/w) or less, but rather because they are minor in the sense that they improve elegance as opposed to being skin actives.

Table 1 below gives the most preferred wash/cleanser formulation of the present invention:

TABLE 1
Cleanser Formulation (Most Preferred)

| Chemical Name | % (w/w) |
|---|---|
| Water, Purified, USP | 61.5000 |
| Sodium Sulfacetamide, USP | 10.0000 |
| Cocamidopropyl Betaine | 8.0000 |
| Sodium Laureth Sulfate | 12.0000 |
| PEG-60 Almond Triglycerides | 3.0000 |
| PEG 150 Pentaerythrity Tetrastearate & | 5.0000 |
| PEG 6 Caprylic/Capric Glycerides &F | |
| Water (Crothix Liquid) | |
| Methylparaben | 0.2000 |
| Edetate Disodium | 0.1500 |
| Sodium Thiosulfate | 0.1500 |

The wash/cleanser generally will have a pH within the range of 6.0 to 9.0, preferably 6.5 to 8.5. The composition of Table 1 has a pH of 7.4. The cleanser/wash viscosity will generally be within the range of 2,000 cps to 25,000 cps, preferably 4,000 cps to 20,000 cps. The composition of Table 1 has a viscosity of 11,000 cps. Representative ranges of broad, preferred and target or presently best known percentage levels of delivery systems for aerosol foams, gels, lotions and creams are shown in Tables 2, 3, 4, 5 and 6.

TABLE 2
Sulfacetamide Sodium Aerosol Foam

| | | target % | Broad % | Pre- |
|---|---|---|---|---|
| | Function | (w/w) | (w/w) | ferred % |
| Active Ingredient: | | | | |
| Sulfacetamide | | | | |
| Sodium 10% | | | | |
| Inactive | | | | |
| Ingredients: | | | | |
| Glycerin | Humectant | 0.1 | 0.0-10 | 0.05-5.0 |
| Cocamidopropyl | Surfactant | 0.5 | 0.1-5.0 | 0.2-3.0 |
| Betaine | | | | |
| Povidone | Film former | 1.0 | 0.05-3.0 | 0.1-2.0 |
| Quaternium-26 & | Surfactant/ | 0.1 | 0.005-2.0 | 0.05-1.0 |
| Propylene Glycol | Skin cond. | | | |
| PVP/DMAPA/ | Film former | 2.5 | 0.1-5.0 | 1.0-4.0 |
| Acrylates | | | | |
| Copolymer | | | | |
| Purified Water | Vehicle | 85.3 | 50.0-90.0 | 55.0-87.0 |
| Lactic Acid | pH adjuster | qs pH | | |
| Sodium Thiosulfate | Antioxidant | 0.15 | 0.05-2.0 | 0.05-1.0 |
| Edetate Disodium | Chelating | 0.15 | 0.05 to 2.0 | 0.05-1.0 |
| | agent | | | |
| Methylparaben | Preservative | 0.20 | 0.05 to 1.0 | 0.08-0.25 |

TABLE 3
Sulfacetamide Sodium Gel

| | | target % | Broad % | Preferred % |
|---|---|---|---|---|
| | Function | (w/w) | (w/w) | (w/w) |
| Active Ingredient: | | | | |
| Sulfacetamide | | | | |
| Sodium 10% | | | | |
| Inactive | | | | |
| Ingredients: | | | | |
| Glycerin | Humectant | 5.0 | 0.0-15 | 1.0-10.0 |
| Quaternium-26 & | Surfactant/ | 0.1 | 0.005-2.0 | 0.05-1.0 |
| Propylene Glycol | skin cond. | | | |
| Xanthan Gum | Thickener | 1.5 | 0.1-5.0 | 0.5-3.0 |
| Purified Water | Vehicle | 82.9 | 50.0-90.0 | 55.0-87.0 |
| Lactic Acid | pH adjuster | qs pH | | |
| Sodium | Antioxidant | 0.15 | 0.05-2.0 | 0.05-1.0 |
| Thiosulfate | | | | |
| Edetate Disodium | Chelating | 0.15 | 0.05 to 2.0 | 0.05-1.0 |
| | agent | | | |
| Methylparaben | Preservative | 0.20 | 0.05 to 1.0 | 0.08-.25 |

TABLE 4
Sulfacetamide Sodium Lotion

| | | target % | Broad % | Preferred % |
|---|---|---|---|---|
| | Function | (w/w) | (w/w) | (w/w) |
| Active Ingredient: | | | | |
| Sulfacetamide | | | | |
| Sodium 10% | | | | |
| Inactive | | | | |
| Ingredients: | | | | |
| Glycerin | Humectant | 5.0 | 0.0-15 | 1.0-10.0 |
| Quaternium-26 & | Surfactant/ | 0.1 | 0.005-2.0 | 0.05-1.0 |
| Propylene Glycol | skin cond. | | | |
| Xanthan Gum | Thickener | 0.5 | 0.0-5.0 | 0.1-2.0 |
| Purified Water | Vehicle | 83.9 | 50.0-90.0 | 55.0-87.0 |
| Lactic Acid | pH adjuster | qs pH | | |
| Sodium | Antioxidant | 0.15 | 0.05-2.0 | 0.05-1.0 |
| Thiosulfate | | | | |
| Edetate Disodium | Chelating agent | 0.15 | 0.05 to 2.0 | 0.05-1.0 |
| Methylparaben | Preservative | 0.20 | 0.05 to 1.0 | 0.08-0.25 |

TABLE 5
Sulfacetamide Sodium Cream

| | | target % | Broad % | Preferred % |
|---|---|---|---|---|
| | Function | (w/w) | (w/w) | (w/w) |
| Active Ingredient: | | | | |
| Sulfacetamide | | | | |
| Sodium 10% | | | | |
| Inactive | | | | |
| Ingredients: | | | | |
| Glycerin | Humectant | 5.0 | 0.0-20.0 | 1.0-15.0 |
| Quaternium-26 & | Surfactant/ | 0.1 | 0.005-2.0 | 0.05-1.0 |
| Propylene Glycol | skin cond. | | | |
| Purified Water | Vehicle | 70.9 | 40.0-90.0 | 50.0-85.0 |
| Emulsifying Wax | Surfactant/ | 2.0 | 0.1-10.0 | 0.5-7.0 |
| | Emulsifier | | | |
| Incroquat | Surfactant/ | 2.5 | 0.1-10.0 | 0.5-7.0 |
| Behenyl TMS | Emulsifier | | | |
| Cetyl Alcohol | Emollient | 2.5 | 0.1-10.0 | 0.5-7.0 |
| Stearyl Alcohol | Emollient | 2.5 | 0.1-10.0 | 0.5-7.0 |
| Sunflower Oil | Emollient | 4.0 | 0.1-15.0 | 0.5-10.0 |
| Sodium | Antioxidant | 0.15 | 0.05-2.0 | 0.05-1.0 |
| Thiosulfate | | | | |
| Edetate Disodium | Chelating | 0.15 | 0.05 to 2.0 | 0.05-1.0 |
| | agent | | | |
| Methylparaben | Preservative | 0.20 | 0.05 to 1.0 | 0.08-0.25 |

TABLE 6
Sulfacetamide Sodium Cream

| | | target % | Broad % | Preferred % |
|---|---|---|---|---|
| | Function | (w/w) | (w/w) | (w/w) |
| Active | | | | |
| Ingredient: | | | | |
| Sulfacetamide | | | | |
| Sodium 10% | | | | |
| Inactive | | | | |
| Ingredients: | | | | |
| Glycerin | Humectant | 10.0 | 0.0-20.0 | 1.0-15.0 |
| Quaternium-26 & | Surfactant/ | 0.1 | 0.005-2.0 | 0.05-1.0 |
| Propylene Glycol | skin cond. | | | |
| Purified Water | Vehicle | 61.7 | 40.0-90.0 | 50.0-85.0 |
| Cetearyl Alcohol/ | Surfactant/ | 2.5 | 0.1-10.0 | 0.5-7.0 |
| Ceteareth 20 | Emulsifier | | | |
| PEG 100 Stearate | Surfactant/ | 1.0 | 0.1-10.0 | 0.5-7.0 |
| | Emulsifier | | | |
| Cetyl Alcohol | Emollient | 2.0 | 0.1-10.0 | 0.5-7.0 |
| Glyceryl | Emollient | 1.0 | 0.1-10.0 | 0.5-7.0 |
| Stearate | | | | |
| Mineral Oil | Emollient | 10.0 | 0.1-20.0 | 0.5-15.0 |
| Dimethicone | Emollient | 0.50 | 0.1-5.0 | 0.2-1.0 |
| Sodium | Antioxidant | 0.15 | 0.05-2.0 | 0.05-1.0 |
| Thiosulfate | | | | |
| Methylparaben | Preservative | 0.25 | 0.05-2.0 | 0.05-1.0 |
| Propylparaben | Preservative | 0.05 | 0.01-0.1 | 0.02-0.07 |
| Phenoxyethanol | Preservative | 0.75 | 0.05-1.0 | 0.08-0.25 |

The following example is offered to illustrate but not limit the invention.

### EXAMPLE

The formulation of Table 1 was used in the following study for determining effectiveness of the short contact time cleaning wash of the present invention.

This study was conducted as a multi-center, open-label study involving patients with seborrheic dermatitis of the face. Specific inclusion/exclusion criteria included: male and female patients 18 years of age and over with seborrheic dermatitis of the facial area.

If the investigator determined that the patient required further treatment after an initial 10 day treatment period, the patient was continued after at least a one day wash-out period.

The individual symptoms of erythema, scaling and roughness of different areas of the face (central forehead, eyebrows, eye area, jaw-cheek area, nasolabial folds, nasal creases, and the central chin area) were rated by the investigator according to a 4-point scale (0 to 3). A global severity score, according to a 5-point (none, minimal, mild, moderate and severe) scale, was also used by the investigator to evaluate each patient. The patient rated erythema, scaling and roughness, however, he/she evaluated the entire facial area (as opposed to the 7 different areas evaluated by the investigator). Subjective symptoms such as burning, stinging and itching were also assessed by each patient. A global improvement rating and product questionnaire was collected from the patient upon study completion. The overall assessment conclusion is here reported.

Patients were instructed to use the product twice daily according to labeling. Patients were instructed to wet the skin, liberally apply product to the areas to be cleansed (forehead, chin, cheeks, nose, eyebrows, etc.), massage gently into skin for 10-20 seconds working into a full lather, rinse thoroughly and pat dry. If the product dried during this process, the cleanser was rinsed off sooner or used less often. Rinsing with plain water removed any excess medication. Repeat applications were allowed according to the investigator's judgment. In mild cases involving the scalp, including no inflammatory types with scaling (dandruff), the wash may have been used to shampoo the patient's hair as directed by the physician.

Table 7 below, shows the areas of the body affected with seborrhea for the treated patients.

TABLE 7
Areas of Body with Seborrhea

| | Number of | Percent of |
|---|---|---|
| Facial Involvement | Patients | Patients |
| Central Forehead | 31 | 72% |
| Eyebrows | 38 | 88% |
| Eye Area | 13 | 30% |
| Jaw and Cheek Area | 25 | 58% |
| Nasolabial Folds | 27 | 63% |
| Nasal Creases | 30 | 70% |
| Central Chin Area | 24 | 56% |
| Other Locations: | | |
| Scalp | 24 | 56% |
| Ears | 18 | 42% |
| Axillae | 2 | 5% |
| Presternal area | 7 | 16% |
| Mid-line of the back | 2 | 5% |
| Arms or legs | 3 | 7% |
| Groin and private areas | 1 | 2% |
| Chest | 1 | 2% |
| Neck | 3 | 7% |

A total of 43 patients were enrolled in this study. All of the patients that completed one or two courses of therapy were cleared (40, 98%) or did not require further therapy (1; 2%). Two patients (4.7%) were prematurely discontinued, 11 (25.6%) patients only required one course of therapy and 30 (69.8%) patients required two courses of therapy to clear their condition completely.

Patients were advised to follow the dosage regime earlier described herein. Thorough rinsing was emphasized. These reports ended with the successful treatment. No patients reported irritation of the eyes.

From the above it can be seen that of the 41 patients that completed the study, 40 (98%) of the patients were considered cleared, and that the composition accomplishes all of its stated objectives.

What is claimed is:

1. A method of treating seborrehic dermatitis, comprising:
topically applying a water rinseable sulfacetamide composition containing no free sulfur to the affected skin area;
allowing the composition to remain in skin contact for a short but therapeutically effective time; and
water rinsing the composition from the affected skin area.

2. The method of claim 1 wherein the short but therapeutically effective time is from ten (10) seconds to two (2) minutes.

3. The method of claim 2 wherein the method is practiced twice a day

4. The method of claim 1 wherein sulfacetamide composition is an alkaline earth metal salt of sulfacetamide.

5. The method of claim 4 wherein the sulfacetamide salt is sulfacetamide sodium.

6. The method of claim 5 wherein the amount of sulfacetamide salt is from 2% (w/w) to 15% (w/w).

7. The method of claim 6 wherein the amount of sulfacetamide salt is from 8% (w/w) to 12% (w/w).

8. The method of claim 7 wherein the amount of sulfacetamide salt is 10% (w/w).

9. The method of claim 1 wherein the composition is in a delivery form selected from the group consisting of washes, aerosol foams, gels, lotions, soap bars, and creams.

10. A water rinseable topical composition for treating seborrehic dermatitis, said composition being free of elemental sulfur, comprising from about 2% (w/w) to about 15% (w/w) of a sulfacetamide composition;
from about 2% (w/w) to about 50% (w/w) of a cleansing surfactants;
pharmaceutical minors selected from the group consisting of detergents foam booster, thickeners, emollients, moisturizers, antioxidants, anti-microbials; and,
the balance being water.

11. The topical composition of claim 10 wherein the sulfacetamide composition is an alkaline earth metal salt of sulfacetamide.

12. The topical composition of claim 11 wherein the sulfacetamide salt is the sodium salt.

13. The topical composition of claim 12 wherein the sulfacetamide salt is present at a level of from about 8% (w/w) to about 12% (w/w).

14. The composition of claim 10 wherein the cleansing surfactant is present at a level of from 5% (w/w) to 20% (w/w).

15. A topical composition for treating seborrehic dermatitis, said composition being water rinseable and free of elemental sulfur, comprising:
(a) a therapeutically effective amount of sulfacetamide sodium;
(b) a pharmaceutical delivery system selected from the group consisting water rinseable washes, aerosol foams, gels, lotions, soap bars, and creams; and
(c) pharmaceutical minors for preserving and enhancing elegance.

16. A composition of claim 15 which is a wash.

17. A composition of claim 16 wherein the wash delivery system includes cleansing and foaming surfactants.

18. A composition of claim 17 wherein the wash delivery system contains as pharmaceutical minors, skin feel enhancers, antioxidants, antimicrobials and a thickener.
